(19) United States
(12) Patent Application Publication
(10) Pub. No.: US 2007/0060621 A1
(43) Pub. Date: Mar. 15, 2007

(54) Methods and compositions for the administration of prodrugs of proton pump inhibitors

(76) Inventor: Patrick M. Hughes, Aliso Viejo, CA (US)

Correspondence Address: ALLERGAN, INC., 2525 DUPONT DRIVE, T2-7H, IRVINE, CA, 92612-1599, US

(21) Appl. No.: 10/554,151

(22) PCT Filed: Jan. 13, 2005
(86) PCT No.: PCT/US05/01297

§ 371 Date: Oct. 19, 2005

Related U.S. Application Data
(60) Provisional application No. 60/545,777, filed on Feb. 18, 2004.

(51) Int. Cl. A61K 31/4439; C07D 403/02

(52) U.S. Cl. 514/338; 546/273.7

(57) ABSTRACT

Disclosed herein are methods, compositions, and dosage forms related to prodrugs of a proton pump inhibitors wherein said compositions and dosage forms do not comprise a salt of phosphoric acid. Principles related to the use of various anions and buffers in relation to these prodrugs are also disclosed.

### CROSS REFERENCE TO RELATED APPLICATIONS

This is a national stage application under 35 U.S.C. § 371 of PCT application PCT/US2005/001297, filed on Jan. 13, 2005, which claims the benefit of Provisional Application No. 60/545,777, filed on Feb. 18, 2004.

### BACKGROUND OF THE INVENTION

1. Field of the Invention

2. Description of the Related Art

Benzimidazole derivatives intended for inhibiting gastric acid secretion are disclosed in U.S. Pat. Nos. 4,045,563; 4,255,431; 4,628,098; 4,686,230; 4,758,579; 4,965,269; 5,021,433; 5,430,042 and 5,708,017. Generally speaking, the benzimidazole-type inhibitors of gastric acid secretion are believed to work by undergoing a rearrangement to form a thiophilic species which then covalently binds to gastric H,K-ATPase, the enzyme involved in the final step of proton production in the parietal cells, and thereby inhibits the enzyme. Compounds which inhibit the gastric H,K-ATPase enzyme are generally known in the field as “proton pump inhibitors” (PPI).

Some of the benzimidazole compounds capable of inhibiting the gastric H,K-ATPase enzyme have found substantial use as drugs in human medicine and are known under such names as LANSOPRAZOLE (U.S. Pat. No. 4,628,098), OMEPRAZOLE (U.S. Pat. Nos. 4,255,431 and 5,693,818), ESOMEPRAZOLE (U.S. Pat. No. 6,369,085) PANTOPRAZOLE (U.S. Pat. No. 4,758,579), and RABEPRAZOLE (U.S. Pat. No. 5,045,552). Some of the diseases treated by proton pump inhibitors and specifically by the five above-mentioned drugs include peptic ulcer, heartburn, reflux esophagitis, erosive esophagitis, non-ulcer dyspepsia, infection by Helicobacter pylori, alrynitis and asthma.

Whereas the proton pump inhibitor type drugs represent a substantial advance in the field of human and veterinary medicine, they are not totally without shortcomings or disadvantages. For example, it is believed that the short systemic half-life of the drug limits the degree of gastric acid suppression currently achieved. Furthermore, it appears that the short plasma half-life of the drug may contribute to significant gastric pH fluctuations that occur several times a day in patients undergoing PPI therapy. Additionally, PPIs are acid-labile, and in most cases it is necessary to enterically coat the drug in order to prevent the acidic milieu of the stomach from destroying the drug before the drug is absorbed into systemic circulation. Thus, any contribution that might improve the acid stability or plasma half-life of the presently used proton pump inhibitors will be a significant improvement in the art.

As further pertinent background to the present invention, applicants note the concept of prodrugs which is well known in the art. Generally speaking, prodrugs are derivatives of per se drugs, which after administration undergo conversion to the physiologically active species. The conversion may be spontaneous, such as hydrolysis in the physiological environment, or may be enzyme catalyzed. From among the voluminous scientific literature devoted to prodrugs in general, the foregoing examples are cited: Design of Prodrugs (Bundgaard H. ed.) 1985 Elsevier Science Publishers B. V. (Biomedical Division), Chapter 1; Design of Prodrugs: Bioreversible derivatives for various functional groups and chemical entities (Hans Bundgaard); Bundgaard et al. Int. J. of Pharmaceutics 22 (1984) 45-56 (Elsevier); Bundgaard et al. Int. J. of Pharmaceutics 29 (1986) 19-28 (Elsevier); Bundgaard et al. J. Med. Chem. 32 (1989) 2503-2507 Chem. Abstracts 93, 137935y (Bundgaard et al.); Chem. Abstracts 95, 138493f (Bundgaard et al.); Chem. Abstracts 95, 138592n (Bundgaard et al.); Chem. Abstracts 110, 57664p (Alminger et al.); Chem. Abstracts 115, 64029s (Buur et al.); Chem. Abstracts 115, 189582y (Hansen et al.); Chem. Abstracts 117, 14347q (Bundgaard et al.); Chem. Abstracts 117, 55790x (Jensen et al.); and Chem. Abstracts 123, 17593b (Thomse\hat{n} et al.).

A publication by Sih., et al. (Journal of Medicinal Chemistry, 1991, vol. 34, pp 1049-1062), describes N-acyloxyalkyl, N-alkoxycarbonyl, N-(aminoethyl), and N-alkoxyalkyl derivatives of benzimidazole sulfoxide as prodrugs of proton-pump inhibitors. According to this article these prodrugs exhibited improved chemical stability in the solid state and in aqueous solutions, but had similar activity or less activity than the corresponding parent compounds having a free imidazole N—H group

U.S. Pat. No. 6,093,734 and PCT Publication WO 00109498 (published on Feb. 24, 2000) describe prodrugs of proton pump inhibitors which include a substituted arylsulfonyl moiety attached to one of the benzimidazole nitrogens of proton pump inhibitors having the structure identical with or related to proton pump inhibitor drugs known by the names LANSOPRAZOLE, OMEPRAZOLE, PANTOPRAZOLE and RABEPRAZOLE.

PCT Publication WO 02/30920 describes benzimidazole compounds which are said to have gastric acid secretion inhibitory and anti H. pylori effects. PCT Publication WO 02/00166 describes compounds that are said to be nitric oxide (NO) releasing derivatives of proton pump inhibitors of the benzimidazole structure.

U.S. patent application having the title “PRODRUGS OF PROTON PUMP INHIBITORS”, filed Jul. 15, 2003 by applicants Michael E. Garst, George Sachs, and Jai M. Shin, which has not yet been assigned a serial number, discloses prodrugs of the proton pump inhibitor type drugs having an arylsulfonyl group with an acidic functional group attached, which provided improved solubility in physiological fluids and improved cell penetration.

### BRIEF DESCRIPTION OF THE INVENTION

Disclosed herein are dosage forms comprising a prodrug of a proton pump inhibitor comprising a biological leaving group bonded to a nitrogen atom of a benzimidazole moiety of said proton pump inhibitor, wherein said dosage form does not comprise a salt of phosphoric acid, and wherein conversion of said prodrug to said proton pump inhibitor depends upon cleavage of a sulfonyl bond.

Also disclosed herein is a method of reducing gastric acid secretion comprising administering to a mammal an effective amount of a sulfonyl prodrug of a proton pump inhibitor in a composition suitable for said administration, provided said composition does not comprise a phosphate buffer.

The use of a sulfonyl prodrug of a proton pump inhibitor for the manufacture of a medicament for the reduction of gastric acid secretion, wherein said medicament does not comprise a phosphate buffer is also disclosed herein.

A pharmaceutical product comprising a composition comprising sulfonamide prodrug of a proton pump inhibitor, and a package for dispensing or storing said prodrug, wherein said composition does not comprise an anionic buffer, is also disclosed herein.

### BRIEF DESCRIPTION OF THE DRAWING FIGURES

FIG. 1 is a plot of the % of the original concentration of compound remaining over time. The original concentration of compound 1 was 0.02 mg/mL, and stability was assessed at 25° C. in 1) water, 2) NaCl salt (μ=0.15), 3) NaCl salt (μ=0.5), 4) phosphate buffer (pH 7.0, μ=0.15), and 5) phosphate buffer (pH 7.0, μ=0.5).

FIG. 2 is a log plot of the data of FIG. 1.

### DETAILED DESCRIPTION OF THE INVENTION

While not intending to limit the scope of the invention in any way, or to be bound in any way by theory, we have surprisingly discovered that monovalent, divalent, and trivalent phosphate ions, and/or phosphate buffers significantly destabilize the prodrug compounds disclosed herein. In other words PO₄³⁻, HPO₄²⁻, H₂PO₄⁻, and/or buffers consisting of combinations of these ions, have an adverse effect upon the stability of prodrugs of proton pump inhibitors contemplated herein. While not intending to be bound in any way by theory, the aqueous stability of the prodrugs disclosed herein is also believed to be relevant to the stability of solid compositions comprising the prodrugs due to the hygroscopic nature of the compounds.

The term “prodrug” has the meaning previously described herein, and in relation to this disclosure refers to a prodrug of a proton pump inhibitor. The term “proton pump inhibitor” also has the meaning previously described herein.

The term “dosage form” used in relation to this invention should be interpreted to mean any form of solid or liquid, or combination thereof, which is intended to be administered to a person, including solutions, suspensions, emulsions, and combinations thereof.

While not intending to limit the scope of the invention in any way, or to bound in any way by theory, it is believed that phosphate may act as a nucleophile, which attacks the sulfonyl moiety of the prodrug, and thus catalyzes the cleavage of the S—N bond, resulting in the formation of the parent PPI compound. As a result, it is believed that other polyvalent anions may also destabilize the prodrugs disclosed herein. Therefore, certain embodiments relate to dosage forms or compositions which do not comprise a polyvalent anion. The term “polyvalent anion” has the term generally understood by those of ordinary skill in the art, i.e. a polyvalent anion is an ion having a charge more negative than −1, e.g. −2, −3, −4, etc.

While not intending to be bound in any way by theory, it is believed that the sulfonyl group, which is derived from a hard acid, may be more susceptible to attack by hard polyvalent anions, according to the generally known and accepted theory related to the reactivity of hard and soft ions. Additionally, hard ions, being more compact, are less likely to be influenced by steric repulsions in approaching the sulfonyl group, the sulfur atom of which has four ligands. Hardness in many cases may be related to the molecular mass of an ion, as seen by the table below, where the harder ions such as carbonate, phosphate, and sulfate, have lower molecular masses than the softer ions. Additionally, smaller ions, regardless of hardness are more likely to destabilize the prodrugs disclosed herein due to the lower susceptibility to unfavorable steric interactions with the sulfonyl group.

| | Molecular Mass |
|---|---|
| Ion | (−1 ion) |
| Carbonate | 61 |
| Phosphate | 97 |
| Sulfate | 97 |
| Malonate | 103 |
| Succinate | 117 |
| Tartrate | 149 |
| Citrate | 191 |

Thus, certain embodiments relate to the molecular mass of an ion. The term “molecular mass” has the meaning generally understood in the art, that is, it is the sum of the atomic masses of all individual atoms in a molecule or ion. For the purposes of this disclosure, the term molecular mass is also applicable to ions consisting of only one atom. In one embodiment the prodrug is in a dosage form or a composition which does not comprise a polyvalent anion having a molecular mass of 100 or less. In another embodiment the prodrug is in a dosage form or a composition which does not comprise a polyvalent anion having a molecular mass of 102 or less. In another embodiment the prodrug is in a dosage form or a composition which does not comprise a polyvalent anion having a molecular mass of 110 or less. In another embodiment the prodrug is in a dosage form or a composition which does not comprise a polyvalent anion having a molecular mass of 120 or less.

Certain embodiments also relate to the solubility of an ion. While not intending to be bound in any way by theory, it is believed that a more soluble anion is more likely to contribute to the instability of the prodrug since a higher concentration of the anion can be present in an aqueous environment, thus increasing the kinetic instability of the compound. The “solubility” as used herein in relation to the concentration of the ion is the concentration of the ion in water when the ion is saturated. Since solubility is dependent upon other components present in a composition, for the purposes of the claim elements, the “solubility” is the concentration of the anion in water when the entire composition in which the anion is present is intimately contacted with water, and the water is saturated with the anion.

In one embodiment the prodrug is in a dosage form or a composition which does not comprise a polyvalent anion having an aqueous solubility of 0.2 M or greater. In another embodiment the prodrug is in a dosage form or a composition which does not comprise a polyvalent anion having an aqueous solubility of 0.15 M or greater. In another embodiment the prodrug is in a dosage form or a composition which does not comprise a polyvalent anion having an aqueous solubility of 0.1 M or greater. In another embodiment the prodrug is administered in a dosage form or a composition which does not comprise a polyvalent anion having an aqueous solubility of 0.02 M or greater. In another embodiment the prodrug is in a dosage form or a composition which does not comprise a polyvalent anion having an aqueous solubility of 0.015 M or greater. In another embodiment the prodrug is in a dosage form or a composition which does not comprise a polyvalent anion having an aqueous solubility of 0.01 M or greater.

In one embodiment the prodrug is in a dosage form or a composition which does not comprise an anion having an aqueous solubility of 0.1 M or greater and a molecular mass of 110 or less. In another embodiment the prodrug is in a dosage form or a composition which does not comprise an anion having an aqueous solubility of 0.01 M or greater and a molecular mass of 110 or less. In another embodiment the prodrug is in a dosage form or a composition which does not comprise an anion having an aqueous solubility of 0.15 M or greater and a molecular mass of 120 or less. In another embodiment the prodrug is in a dosage form or a composition which does not comprise an anion having an aqueous solubility of 0.015 M or greater and a molecular mass of 120 or less.

In one embodiment the prodrug is in a dosage form or a composition which does not comprise an anionic buffer. The term “buffer” as used herein should be construed to have a narrow meaning according to that which is generally understood in the art. That is, not only should the “buffer” have one or more of the required components which make it a buffer, but the buffer should be at such a concentration as to be effective in maintaining the pH at the desired value. A phosphate buffer is a combination of phosphoric acid and its salts in a ratio and at an effective concentration, such that the pH is maintained at its desired value for as long as necessary. The desired value of the pH and the amount of time that the pH must be maintained at that value are dependent upon the composition or dosage form in which the drug is present. Such a determination can be readily made by a person of ordinary skill in the art.

Another embodiment comprises a dosage form or composition comprising a prodrug and a buffer which is not anionic. Buffers which are not anionic include zwitterionic buffers comprising amino acids such as glycine, or other zwitterionic species such as betaines, and cationic buffers including amines such as triethanolamine or diethanolamine and their salts.

In one embodiment the prodrug is in a dosage form or a composition which does not comprise more than 0.1 moles of a polyvalent anion for every 1 mole of said prodrug, wherein the polyvalent anion has an aqueous solubility of 0.1 M or greater. In another embodiment the prodrug is in a dosage form or a composition which does not comprise more than 0.05 moles of a polyvalent anion for every 1 mole of said prodrug, wherein said polyvalent anion has an aqueous solubility of 0.15 M or greater.

The term “biological leaving group” as used herein refers to a moiety which is cleaved from the remainder of the molecule in the body of a mammal such that the remainder of the molecule is a proton pump inhibitor, or is readily converted to a proton pump inhibitor by a process such a protonation; deprotonation; quenching of an unstable intermediate such as a radical, radical ion, carbocation, carbene, or nitrene; tautomerization; or a similar process. In one embodiment the, biological leaving group comprises a sulfonyl group, where the sulfur atom is directly bonded to the nitrogen atom of the benzimidazole moiety. A “sulfonyl” moiety or group is defined herein as a moiety comprising an SO₂ group, where a sulfur atom is directly covalently bonded to two oxygen atoms. A “sulfonyl bond” is a bond between the sulfur of the sulfonyl group and another atom. In another embodiment, the biological leaving group comprises a sulfonyl group and an aromatic ring, wherein the sulfur atom is directly bonded to the nitrogen atom of the benzimidazole moiety. The term “aromatic ring” has the broadest meaning generally understood in the art. In another embodiment, the biological leaving group comprises a phenylsulfonyl group, wherein the sulfur atom is directly bonded to the nitrogen atom of the benzimidazole moiety. The term “phenylsulfonyl” moiety should be broadly interpreted to mean any moiety where the sulfur of the SO₂ group is directly covalently bonded to a carbon that is part of a phenyl ring. The term “phenyl ring” should be broadly understood to mean any ring comprising six carbon atoms having three conjugated double bonds. Thus, a phenylsulfonyl moiety could be monosubstituted, meaning that the sulfonyl group is the only group directly attached to the phenyl ring, or the phenylsulfonyl moiety could have from 1 to 5 additional substituents which are not a hydrogen atom, and are directly attached to a carbon of the phenyl ring.

While not intending to limit the scope of the invention in any way, in many situations one might choose a prodrug which would be converted after administration into one of the widely used and well tested commercially available proton pump inhibitors (PPI) such as lansoprazole, esomeprazole, omeprazole, pantoprazole, and rabeprazole. In situations where one of the commercially available PPIs is used, one may want to consider circumstances related to the individual to which the prodrug is administered in making decisions related to the choice of the compound used. For example, if the person to which the prodrug is being administered is known to respond well to omeprazole, then one may consider using a prodrug of omeprazole as disclosed herein. In another situation, a person may have a history of being effectively treated by lansoprazole, in which case one may consider using a prodrug of lansoprazole as disclosed herein. The specific disclosure related to the proton pump inhibitor is given herein merely to provide guidance and direction to one practicing the disclosure herein, and is not intended to limit the overall scope of the invention in any way.

Certain embodiments relate to particular structures, which are useful as prodrugs.
One embodiment comprises 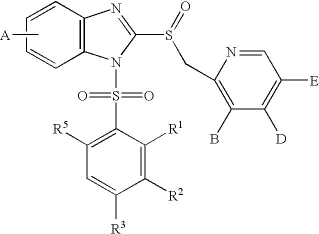
or a pharmaceutically acceptable salt thereof
wherein
A is H, OCH₃, or OCHF₂;
B is CH₃ or OCH₃;
D is OCH₃, OCH₂CF₃, or O(CH₂)₃OCH₃;
E is H or CH₃;
R¹, R², R³, and R⁵ are independently H, CH₃, CO₂H, CH₂CO₂H, (CH₂)₂CO₂H, CH(CH₃)₂, OCH₂C(CH₃)₂CO₂H, OCH₂CO₂CH₃, OCH₂CO₂H, OCH₂CO₂NH₂, OCH₂CONH₂(CH₂)₅CO₂CH₃, or OCH₃.

In another embodiment related to the one just described, R¹, R², R³, and R⁵ are independently H, CH₃, CO₂H, CH₂CO₂H, (CH₂)₂CO₂H, OCH₂CO₂CH₃, OCH₂CO₂H, OCH₂CONH₂(CH₂)₅CO₂CH₃, or OCH₃.

In certain embodiments, the prodrug has a structure comprising 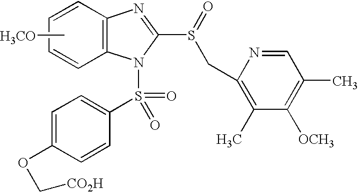
or a pharmaceutically acceptable salt thereof.
Other embodiments comprise 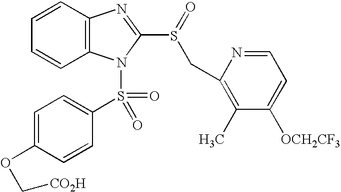
or a pharmaceutically acceptable salt thereof.
Other embodiments comprise 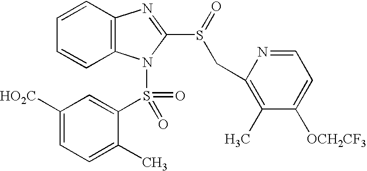
or a pharmaceutically acceptable salt thereof.
Other embodiments comprise 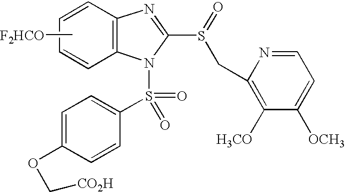
or a pharmaceutically acceptable salt thereof.
Other embodiments comprise 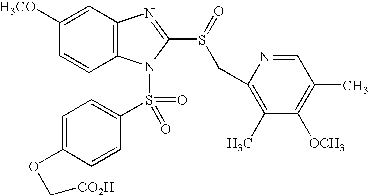
or a pharmaceutically acceptable salt thereof.
Other embodiments comprise 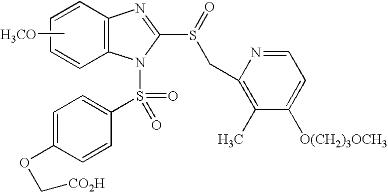
or a pharmaceutically acceptable salt thereof.

A “pharmaceutically acceptable salt” is any salt that retains the activity of the parent compound and does not impart any deleterious or untoward effect on the subject to which it is administered and in the context in which it is administered. Pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

The prodrugs of the present invention can be prepared by the methods described in the following U.S. patent documents, all of which are expressly incorporated by reference herein: U.S. Pat. No. 6,093,734; U.S. patent application Ser. No. 09/783,807, filed Feb. 14, 2001; the U.S. pat. app. having the title “PRODRUGS OF PROTON PUMP INHIBITORS”, filed Jul. 15, 2003 by applicants Michael E. Garst, George Sachs, and Jai M. Shin, which has not yet been assigned a serial number; and the U.S. pat. app. having the title “PROCESS FOR PREPARING ISOMERICALLY PURE PRODRUGS OF PROTON PUMP INHIBITORS”, filed Jul. 15, 2003 by applicants Michael E. Garst, Lloyd J. Dolby, Shervin Esfandiari, Vivian R. Mackenzie, Alfred A. Avey, Jr., David C. Muchmore, Geoffrey K. Cooper, and Thomas C. Malone, which has not yet been assigned a serial number. However, these methods are only given to provide guidance, and are not meant to limit the scope of the invention in any way. One of ordinary skill in the art will recognize that there are many ways in which the prodrugs of the present invention can be prepared without departing from the spirit and scope of the present invention.

Those skilled in the art will readily understand that for oral administration the compounds of the invention are admixed with pharmaceutically acceptable excipients which per se are well known in the art. Specifically, a drug to be administered systemically, it may be confected as a powder, pill, tablet or the like, or as a syrup or elixir suitable for oral administration. Description of the substances normally used to prepare tablets, powders, pills, syrups and elixirs can be found in several books and treatise well known in the art, for example in Remington's Pharmaceutical Science, Edition 17, Mack Publishing Company, Easton, Pa.

Parenteral administration is generally characterized by injection. Injectables can be prepared in conventional forms, either as liquid solutions or suspensions, solid forms suitable for dissolving or suspending in liquid prior to injection, or as emulsions. Descriptions of substances and methods normally used to prepare formulations for parenteral administration can be found in several treatises and books well known in the art such as, Handbook On Injectable Drugs (11th edition), edited by Lawrence A. Trissel, (Chicago: Login Brothers Book Company; Jan. 15, 2001).

The following examples provide guidance and direction in making and using the invention. However, they are not to be interpreted as limiting the scope of the invention in any way.

### EXAMPLE 1

Compounds specifically contemplated in relation to embodiments disclosed herein are presented in Table 1 below. The generic structure, I, is shown as a combination of a proton pump inhibitor (X) and a sulfonyl-bearing moiety which is attached to the proton pump inhibitor to form the prodrug according to the formula below. The identity of each group represented by R¹-R⁵ is shown in the table.

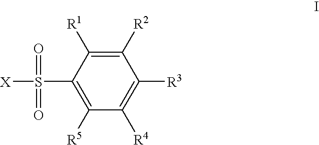

The different possibilities for X are shown below.

TABLE 1

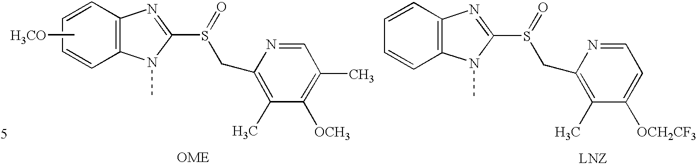

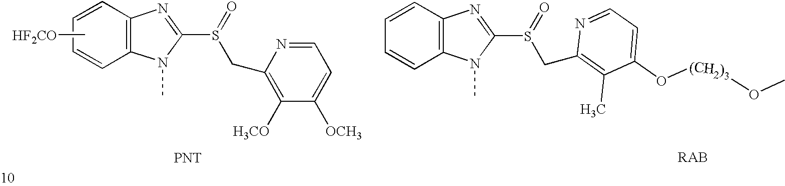

| Compound | X | R¹ | R² | R³ | R⁴ | R⁵ |
|---|---|---|---|---|---|---|
| 1 | OME | H | H | OCH₂CO₂H | H | H |
| 2 | OME | CH₃ | H | OCH₂CO₂H | H | CH₃ |
| 3 | OME | H | H | OCH₂C(CH₃)₂CO₂H | H | H |
| 4 | OME | CH₃ | H | OCH₂C(CH₃)₂CO₂H | H | CH₃ |
| 5 | OME | H | H | CH₂CO₂H | H | H |
| 6 | OME | H | CO₂H | H | H | H |
| 7 | LNZ | H | CO₂H | H | H | H |
| 8 | LNZ | H | CO₂H | OCH₃ | H | H |
| 9 | LNZ | H | H | CH₂CO₂H | H | H |
| 10 | LNZ | H | H | OCH₂CO₂H | H | H |
| 11 | LNZ | H | H | OCH₂C(CH₃)₂CO₂H | H | H |
| 12 | LNZ | H | CH₂CO₂H | CH₂CO₂H | H | H |
| 13 | LNZ | H | CO₂H | H | H | CH₃ |
| 14 | LNZ | H | CO₂H | H | H | OCH₃ |
| 15 | LNZ | CH(CH₃)₂ | H | CH₂CO₂H | H | H |
| 16 | LNZ | H | OCH₂CO₂H | CO₂H | H | H |
| 17 | LNZ | CH(CH₃)₂ | H | OCH₂CO₂H | H | CH₃ |
| 18 | LNZ | H | H | CO₂H | H | H |
| 19 | LNZ | H | (CH₂)₂CO₂H | CH₃ | H | H |
| 20 | OME | H | H | OCH₂CO₂CH₃ | H | H |
| 21 | OME | H | H | OCH₂CO₂NH₂ | H | H |
| 22 | OME | H | CO₂H | CO₂H | H | H |
| 23 | OME | H | CO₂H | OCH₂CO₂H | H | H |
| 24 | OME | H | OCH₂CO₂H | OCH₂CO₂H | H | H |
| 25 | OME | OCH₃ | H | CO₂H | H | H |
| 26 | OME | H | | CO₂H | H | H |
| 27 | OME | H | CO₂H | H | H | CH₃ |
| 28 | PNT | H | H | OCH₂CO₂H | H | H |
| 29 | PNT | H | CO₂H | H | H | CH₃ |
| 30 | RAB | H | CO₂H | H | H | H |
| 31 | RAB | H | CO₂H | H | H | CH₃ |
| 32 | RAB | CH₃ | H | OCH₂CO₂H | H | CH₃ |
| 33 | RAB | H | H | CO₂H | H | H |
| 34 | LNZ | CH₃ | H | OCH₂CO₂H | H | CH₃ |
| 35 | LNZ | H | OCH₂CO₂H | OCH₂CO₂H | H | H |
| 36 | LNZ | H | H | CO₂H | H | H |
| 37 | LNZ | CH₃ | H | CO₂H | H | H |
| 38 | LNZ | H | (CH₂)₂CO₂H | OCH₃ | H | H |
| 39 | OME | CH₃ | H | OCH₂CONH₂(CH₂)₅ | H | CH₃ |
| | | | | CO₂CH₃ | | |
| 40 | OME | H | H | OCH₂CONH₂(CH₂)₅ | H | H |
| | | | | CO₂CH₃ | | |
| 41 | OME | H | H | (CH₂)₂CO₂H | H | H |
| 42 | OME | H | (CH₂)₂CO₂H | OCH₃ | H | H |

These compounds have been prepared according to procedures described the U.S. pat. app. having the title “PRODRUGS OF PROTON PUMP INHIBITORS”, filed Jul. 15, 2003 by applicants Michael E. Garst, George Sachs, and Jai M. Shin, which has not yet been assigned a serial number; and the U.S. pat. app. having the title “PROCESS FOR PREPARING ISOMERICALLY PURE PRODRUGS OF PROTON PUMP INHIBITORS”, filed Jul. 15, 2003 by applicants Michael E. Garst, Lloyd J. Dolby, Shervin Esfandiari, Vivian R. Mackenzie, Alfred A. Avey, Jr., David C. Muchmore, Geoffrey K. Cooper, and Thomas C. Malone, which has not yet been assigned a serial number, incorporated by reference previously herein. These aforementioned patent documents, as well as the provisional U.S. Patent Application No. 513880, filed on Oct. 22, 2003 by applicants Jie Shen, Devin F. Welty, and Diane D. Tang-Liu, incorporated herein by reference, demonstrate that compounds 1-42 decompose in vivo to form proton pump inhibitors.

### EXAMPLE 2

The physicochemical properties of compound 1 were analyzed. Compound 1 was found to be hygroscopic, in that 9% weight gain was observed for the compound after 14 days of storage at 25° C. at 75% relative humidity.

TABLE 2A
Stability Profile of Compound 1 at 25° C. in Buffered
Aqueous Solutions

| | | Half-life | | Degradation |
|---|---|---|---|---|
| | Buffer | (t_{1/2}) | Shelf life | Rate Constant |
| pH | Composition | hours | (t_{90%}) hours | (k) 1/hours |
| 1 | 0.1 M HCl | 3.6 | 0.5 | 0.194 |
| 3 | Citric Acid (0.1 M)/ | 78.0 | 11.9 | 0.009 |
| | Na₂HPO₄ (0.2 M) | | | |
| 5 | Citric Acid (0.1 M)/ | 89.2 | 13.6 | 0.008 |
| | Na₂HPO₄ (0.2 M) | | | |
| 7 | sodium phosphate | 286.8 | 43.6 | 0.002 |
| | (0.1-0.2 M) | | | |
| 7.4 | sodium phosphate | 291.2 | 44.3 | 0.002 |
| | (0.1-0.2 M) | | | |
| 9 | sodium phosphate | 23.0 | 3.5 | 0.030 |
| | (0.1-0.2 M) | | | |
| 10 | sodium phosphate | 2.3 | 0.4 | 0.298 |
| | (0.1-0.2 M) | | | |
| | No buffer | 2863.6 | 435.4 | 0.0002 |

The aqueous stability data of compound 1 is presented in Table 2B. These results show that, the half-life (t_{1/2}), the shelf-life (t_{90%}), and the rate constant for degradation (k) for compound 1 are significantly improved at the pH values of 7 and 7.4 relative to the other pH values studied. While not intending to be bound in any way by theory, the fact that compound 1 becomes less stable in both acidic and basic environments, points to both acid and base-catalyzed degradation of these compounds. The base-catalyzed degradation is unexpected because the commercial proton pump inhibitors are stabilized in aqueous solutions by adjusting the solution to high pH. In fact, while not intending to be bound or limited in any way by theory, compound 1 appears to be more susceptible to base-catalyzed degradation than acid-catalyzed degradation, since its half-life is longer at pH 5, where the H⁺ concentration is 10⁻⁵ M than its half-life is at pH 9, where the OH⁻ concentration is 10⁻⁵ M. Similarly, compound 1 is less stable at pH 10, where the OH⁻ concentration is 10⁻⁴ M than it is at pH 1, where the H⁺ concentration is 0.1 M. While not intending to be bound or limited in any way by theory, these results unexpectedly show that the optimum pH for the compounds disclosed herein is around neutral, and that formulation of aqueous dosage forms of near neutral pH should greatly improve the stability of the prodrugs, thus improving shelf-life and facilitating formulation.

While not intending to be bound or limited in any way by theory, based upon the fact that the stability of compound 1 is essentially unchanged from pH 7 to pH 7.4, and based upon the other data presented in Table 2A, it is reasonable to believe that these compounds should be most stable when the pH is from about pH 6.5 to about 8.

Additionally, these results demonstrate that the prodrugs are significantly more stable in neutral aqueous solutions than the proton pump inhibitors. The stability of omeprazole and other proton pump inhibitors have been reported (Kromer et al., “Differences in pH-Dependent Activation Rates of Substituted Benzimidazoles and Biological in vitro Correlates”, Pharmacology 1998; 56:57-70; and Ekpe et al, “Effect of Various Salts on the Stability of Lansoprazole, Omeprazole, and Pantoprazole as Determined by High Performance Liquid Chromatograpy”, Drug Development and Industrial Pharmacy, 25(9), 1057-1065 (1999)), and while the stability is somewhat buffer dependent, typical half-lives for omeprazole are about 40 hours at pH 7, which is nearly an order of magnitude shorter than the prodrug half-live presented in Table 2A. While not intending to be bound in any way by theory, or to limit the scope of the invention in any way, these results suggest that the compounds of disclosed herein can be injected at a more neutral pH than is currently possible with the currently available proton pump inhibitors. This should allow bolus injection of the compounds disclosed herein as opposed to the slow infusion of the drug currently in practice because the present compositions will not have the irritation associated with the high pH traditionally used with proton pump inhibitors. Additionally, while not intending to be bound in any way by theory, or to limit the invention in any way, these results also demonstrate that the aqueous solution can be stored for a longer period of time prior to administration, and that the solid will be easier to handle, because moisture is less likely to destabilize the active compound.

Surprisingly, we found that the unbuffered prodrug had a half-life that was about an order of magnitude longer than the buffered prodrug. This finding was investigated in detail, and the results are presented in the next example.

### EXAMPLE 3

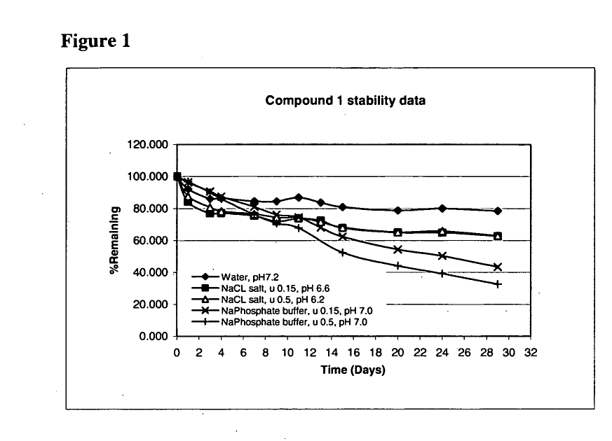

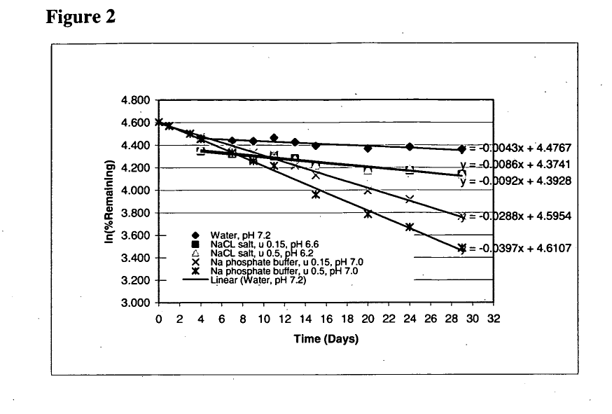

The stability of compound 1 at a concentration of 0.02 mg/mL in water at 25° C. was assessed in 1) water, 2) NaCl salt (μ=0.15), 3) NaCl salt (μ=0.5), 4) phosphate buffer (pH 7.0, μ=0.15), and 5) phosphate buffer (pH 7.0, μ=0.5). For the buffer solutions, the ionic strength (μ) was adjusted using sodium chloride, and the buffer concentration of the two solutions was equal (0.1 M). The amount of remaining compound 1 is presented as the % of the original concentration of 0.02 mg/mL for each sample in Table 3a and in FIG. 1. These results show that beyond four days, the stability of the prodrug in the corresponding environment decreased in the following order: water>NaCl salt>>phosphate buffer. The results of the early measurements are anomalous, and suggest an impurity in the sample that may have affected the stability before the impurity was consumed. FIG. 2, which is a log plot of the remaining sample, clearly shows a first order decay of the sample from 3-29 days, supporting the hypothesis that the decay of the sample of the first three days are anomalous. The half lives of each sample during this time period were determined, and are presented in Table 3b.

TABLE 3a

| | Compound 1% Remaining | | | | |
|---|---|---|---|---|---|
| | | | | Phosphate | Phosphate |
| Sampling | | NaCL, | NaCL, | buffer, | buffer, |
| schedule | Water | u = 0.15 | u = 0.5 | u = 0.15 | u = 0.5 |
| (Days) | (pH 7.2) | (pH 6.6) | (pH 6.2) | (pH 7.0) | (pH 7.0) |
| 1 | 92.212 | 84.134 | 87.598 | 96.294 | 96.709 |
| 3 | 86.085 | 76.960 | 80.750 | 90.597 | 90.087 |
| 4 | 86.410 | 77.037 | 78.348 | 87.434 | 85.994 |
| 7 | 84.569 | 75.398 | 76.513 | 80.995 | 76.165 |
| 9 | 84.452 | 71.861 | 74.482 | 76.176 | 70.768 |
| 11 | 86.930 | 73.763 | 74.312 | 74.520 | 67.826 |
| 13 | 83.661 | 72.390 | 71.691 | 68.020 | n/a |
| 15 | 80.913 | 67.858 | 68.167 | 62.389 | 52.494 |
| 20 | 78.768 | 64.953 | 65.173 | 54.360 | 44.057 |
| 24 | 79.915 | 64.848 | 65.753 | 50.246 | 39.181 |
| 29 | 78.412 | 62.731 | 62.867 | 43.321 | 32.626 |

While not intending to be bound in any way by theory, the fact that the prodrug has a significantly shorter half life and shelf life, and faster decay rate in the phosphate buffer than it had in water at a nearly identical pH demonstrates that phosphate has a destabilizing effect upon the prodrug. While not intending to be bound in any way by theory, it also appears that the presence of other ions may have some adverse effect upon the stability of these compounds, although it is significantly less than that of the phosphate buffer. However, this contribution may simply be a product of the lower pH of the samples.

Decomposition of the prodrug in this and the previous example gave the parent proton pump inhibitor.

TABLE 3b

| | Half Life | Shelf Life (t | Rate Constant |
|---|---|---|---|
| | (t½) | 90%) | (k) |
| Compound 1 sample | days | days | 1/days |
| water, pH 7.2 | 161.163 | 24.502 | 0.0043 |
| NaCl, u = 0.15, pH 6.6 | 80.581 | 12.251 | 0.0086 |
| NaCl, u = 0.5, pH 6.6 | 75.326 | 11.452 | 0.0092 |
| Na phosphate buffer, | 24.063 | 3.658 | 0.0288 |
| u = 0.15, pH 7.0 | | | |
| Na phosphate buffer, | 17.589 | 2.674 | 0.0394 |
| u = 0.5, pH 7.0 | | | |

### EXAMPLE 3

Capsules are prepared according to well-known commercial processes using the composition shown in Table 3.

TABLE 3

| Component | Amount (mg) |
|---|---|
| Compound 1 | 20 |
| Lactose | 200 |
| Magnesium Stearate | 3 |

### EXAMPLE 4

The capsule prepared according to example 3 is orally administered daily to a person suffering from heartburn. Relief of pain begins to occur within about 1 day, and continues as long as the person takes the dosage form.

What is claimed is:

1. A dosage form comprising
a prodrug of a proton pump inhibitor comprising a biological leaving group bonded to a nitrogen atom of a benzimidazole moiety of said proton pump inhibitor,
wherein said dosage form does not comprise a salt of phosphoric acid,
and wherein conversion of said prodrug to said proton pump inhibitor depends upon cleavage of a sulfonyl bond.

2. The dosage form of claim 1 wherein said proton pump inhibitor is selected from the group consisting of omeprazole, esomeprazole, lansoprazole, pantoprazole, and rabeprazole.

3. The dosage form of claim 1 wherein the proton pump inhibitor is omeprazole.

4. The dosage form of claim 1 wherein the biological leaving group comprises an phenyl ring.

5. The dosage form of claim 1 comprising 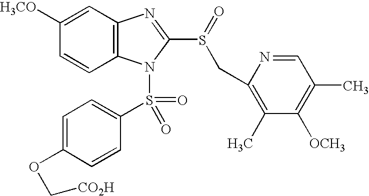
or a pharmaceutically acceptable salt thereof.

6. The dosage form of claim 1 comprising 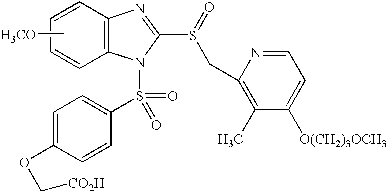
or a pharmaceutically acceptable salt thereof.

7. The dosage form of claim 1 which does not comprise a polyvalent anion having a molecular mass of 100 or less.

8. The dosage form of claim 1 which does not comprise a buffer.

9. The dosage form of claim 1 which does not comprise more than 0.1 moles of a polyvalent anion for every 1 mole of said prodrug, wherein said polyvalent anion has an aqueous solubility of 0.1 M or greater.

10. The dosage form of claim 1 which does not comprise a polyvalent anion having an aqueous solubility of 0.1 M or greater.

11. The dosage form of claim 1 which does not comprise a polyvalent anion having an aqueous solubility of 0.01 M or greater.

12. The dosage form of claim 6 which does not comprise an anion having an aqueous solubility of 0.1 M or greater and a molecular mass of 110 or less.

13. The dosage form of claim 5 which does not comprise an anion having an aqueous solubility of 0.01 M or greater and a molecular mass of 110 or less.

14. The dosage form of claim 1 which is a solid.

15. The dosage form of claim 1 which is a liquid.

16. A method of reducing gastric acid secretion comprising administering to a mammal an effective amount of a sulfonyl prodrug of a proton pump inhibitor in a composition suitable for said administration, provided said composition does not comprise a phosphate buffer.

17. The method of claim 16 wherein the proton pump inhibitor is lansoprazole.

18. The method of claim 16 wherein the proton pump inhibitor is esomeprazole.

19. The method of claim 16 wherein the proton pump inhibitor is omeprazole.

20. The method of claim 16 wherein the proton pump inhibitor is pantoprazole.

21. The method of claim 16 wherein the proton pump inhibitor is rabeprazole.

22. The method of claim 16 wherein said biological leaving group comprises a phenylsulfonyl group, wherein the sulfur atom is directly bonded to the nitrogen atom of the benzimidazole moiety.

23. The method of claim 16 comprising 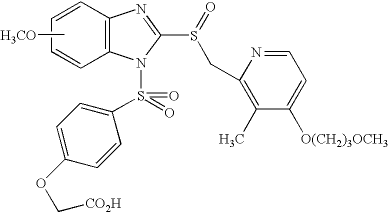
or a pharmaceutically acceptable salt thereof.

24. The method of claim 16 wherein said prodrug is administered in a dosage form or a composition which does not comprise a polyvalent anion having a molecular mass of 102 or less.

25. The method of claim 16 wherein said prodrug is administered in a dosage form or a composition which does not comprise a buffer.

26. The method of claim 16 wherein said prodrug is administered in a dosage form or a composition which does not comprise more than 0.05 moles of a polyvalent anion for every 1 mole of said prodrug, wherein said polyvalent anion has an aqueous solubility of 0.15 M or greater.

27. The method of claim 16 wherein said prodrug is administered in a dosage form or a composition which does not comprise a polyvalent anion having an aqueous solubility of 0.2 M or greater.

28. The method of claim 16 wherein said prodrug is administered in a dosage form or a composition which does not comprise a polyvalent anion having an aqueous solubility of 0.02 M or greater.

29. The method of claim 16 wherein said prodrug is administered in a dosage form or a composition which does not comprise an anion having an aqueous solubility of 0.15 M or greater and a molecular mass of 120 or less.

30. The method of claim 19 wherein said prodrug is administered in a dosage form or a composition which does not comprise an anion having an aqueous solubility of 0.015 M or greater and a molecular mass of 120 or less.

31. A pharmaceutical product comprising
a composition comprising sulfonamide prodrug of a proton pump inhibitor, and
a package for dispensing or storing said prodrug,
wherein said composition does not comprise an anionic buffer.

32. The product of claim 25 comprising 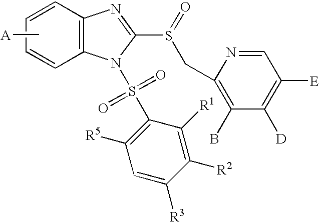
or a pharmaceutically acceptable salt thereof
wherein
A is H, OCH₃, or OCHF₂;
B is CH₃ or OCH₃;
D is OCH₃, OCH₂CF₃, or O(CH₂)₃OCH₃;
E is H or CH₃;
R¹, R², R³, and R⁵ are independently H, CH₃, CO₂H, CH₂CO₂H, (CH₂)₂CO₂H, CH(CH₃)₂, OCH₂C(CH₃)₂CO₂H, OCH₂CO₂CH₃, OCH₂CO₂H, OCH₂CO₂NH₂, OCH₂CONH₂(CH₂)₅CO₂CH₃, or OCH₃.

33. The product of claim 32 wherein R¹, R², R³, and R⁵ are independently H, CH₃, CO₂H, CH₂CO₂H, (CH₂)₂CO₂H, OCH₂CO₂CH₃, OCH₂CO₂H, OCH₂CONH₂(CH₂)₅CO₂CH₃, or OCH₃.

34. The product of claim 31 comprising 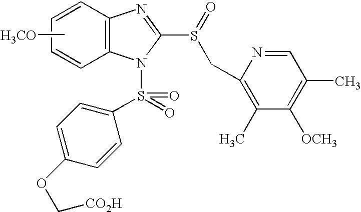
or a pharmaceutically acceptable salt thereof.

35. The product of claim 31 comprising 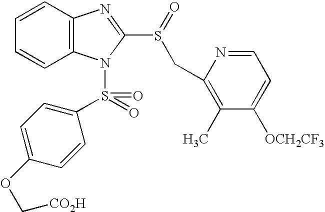
or a pharmaceutically acceptable salt thereof.

36. The product of claim 31 comprising 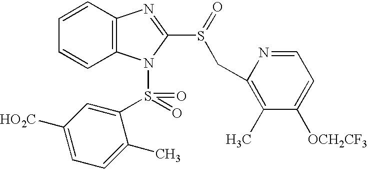
or a pharmaceutically acceptable salt thereof.

37. The product of claim 31 comprising 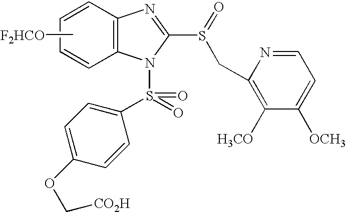
or a pharmaceutically acceptable salt thereof.

38. The product of claim 31 comprising 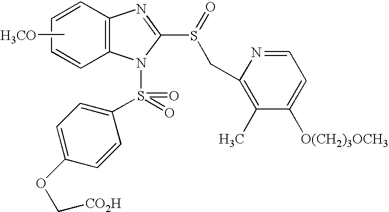
or a pharmaceutically acceptable salt thereof.

39. The product of claim 31 comprising 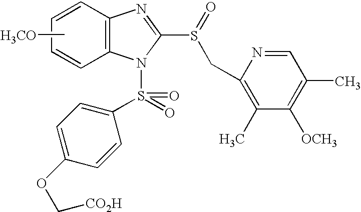
or a pharmaceutically acceptable salt thereof.

40. The dosage form of claim 1 comprising a buffer which is not anionic.

41. The dosage form of claim 1 which is a liquid.

42. The dosage form of claim 1 which is a solution.

43. The dosage form of claim 1 which is a suspension or an emulsion.
